SEQUENCE LISTING (6 sequences)

SEQ ID NO: 1 - DNA, 25 bp, Artificial Sequence
  Forward primer for Atrogin-1
gttactgcaacaaggagaatctgtt

SEQ ID NO: 2 - DNA, 25 bp, Artificial Sequence
  Reverse primer for Atrogin-1
ccgtatgagtcttatgttttgctgg

SEQ ID NO: 3 - DNA, 20 bp, Artificial Sequence
  Forward primer for MuRF-1
ccggacggaaatgctatgga

SEQ ID NO: 4 - DNA, 20 bp, Artificial Sequence
  Reverse primer for MuRF-1
agcctggaagatgtcgttgg

SEQ ID NO: 5 - DNA, 24 bp, Artificial Sequence
  Reverse primer for beta-Actin
tgacaggatgcagaaggagattac

SEQ ID NO: 6 - DNA, 23 bp, Artificial Sequence
  Reverse primer for beta-Actin
taaaacgcagctcagtaacagtc

(19) United States
(12) Patent Application Publication
(10) Pub. No.: US 2022/0088106 A1
(43) Pub. Date: Mar. 24, 2022

(54) COMPOSITION COMPRISING CUDRANIA TRICUSPIDATE AS EFFECTIVE COMPONENT FOR ALLEVIATING, TREATING, OR PREVENTING MUSCULAR DISEASES, OR IMPROVING MUSCULE FUNCTIONS

(71) Applicant: AAT COSTECH CO., LTD., Seoul (KR)

(72) Inventors: Jae Kwan HWANG, Seoul (KR); Chang Hee KIM, Seoul (KR); Ji Hee YOO, Seongnam-si (KR)

(73) Assignee: ATT COSTECH CO., LTD, Seoul (KR)

(21) Appl. No.: 17/276,272

(22) PCT Filed: Jun. 8, 2020
(86) PCT No.: PCT/KR2020/007411

§ 371 Date: Mar. 15, 2021

(30) Foreign Application Priority Data
Jun. 7, 2019 (KR) 10-2019-0067189
Jun. 8, 2020 (KR) 10-2020-0068881

(51) Int. Cl. A61K 36/60; A61K 8/9778; A23L 33/00; A23L 33/105; A23K 10/30

(52) U.S. Cl. CPC A61K 36/60; A61K 8/9778; A23L 33/40; A23L 33/105; A23V 2002/00; A61K 2236/331; A61K 2236/35; A61K 2236/37; A23K 10/30

(57) ABSTRACT

The present disclosure relates to a composition for preventing and treating a muscular disease, or improving muscular function, which contains Cudrania tricuspidata such as a pulverization product or an extract of Cudrania tricuspidata as an active ingredient. The Cudrania tricuspidata according to the present disclosure such as the pulverization product or extract of Cudrania tricuspidata has an effect of increasing the activity of mTOR, which is involved in muscle protein synthesis, and reducing the mRNA expression of MuRF-1 and atrogin-1, which are involved in muscle protein degradation, increases muscle mass and improves muscular function. Therefore, it may be usefully utilized in the prevention, alleviation or treatment of various muscular diseases such as atony, muscular atrophy, muscular dystrophy, muscle degeneration, muscle weakness, cachexia, sarcopenia, etc.

### CROSS-REFERENCE TO RELATED APPLICATIONS

This application is a § 371 national stage entry of International Application No. PCT/KR2020/007411, filed on Jun. 8, 2020, which claims priority to Korean Patent Application No. 10-2019-0067189 filed on Jun. 7, 2019 and Korean Patent Application No. 10-2020-0068881 filed on Jun. 8, 2020, the entire contents of which are incorporated herein by reference.

### SEQUENCE LISTING

The instant application contains a Sequence Listing, which has been submitted electronically on ASCII format and is hereby incorporated by reference in its entirety. Said ASCII text file, created Oct. 26, 2021, is named G1035-19201_SequenceListing is 1,587 bytes in size.

### TECHNICAL FIELD

The present disclosure relates to a composition for alleviating, treating or preventing a muscular disease, or improving muscular function, more particularly to a composition for alleviating, treating or preventing a muscular disease, or improving muscular function, which contains Cudrania tricuspidata such as a pulverization product of Cudrania tricuspidata or a Cudrania tricuspidata extract as an active ingredient.

### BACKGROUND ART

Muscle atrophy refers to the weakness and degeneration of muscle due to gradual decrease in muscle mass (Cell, 119(7): 907-910, 2004). Muscle atrophy is caused by immobility, oxidative stress or chronic inflammation and weakens muscular function and motor ability (Clinical Nutrition, 26(5): 524-534, 2007). The most important factor that determines muscular function is muscle mass, and it is maintained by the balance between protein synthesis and degradation. Muscle atrophy occurs when protein degradation exceeds synthesis (The International Journal of Biochemistry and Cell Biology, 37(10): 1985-1996, 2005).

Muscle size is mediated by intracellular signaling pathways that induce anabolism or catabolism in muscles. Muscle protein synthesis is increased when the signaling that induces the synthesis of muscle protein exceeds the signaling that induces the degradation thereof, resulting in increase of muscle size (hypertrophy) or increase of muscle fibers (hyperplasia) (The Korea Journal of Sports Science, 20(3): 1551-1561, 2011).

The factors involved in muscle protein synthesis induce protein synthesis by phosphorylating downstream proteins upon stimulation of the phosphatidylinositol-3 kinase (PI3K)/Akt pathway in muscle cells. The activation of mammalian target of rapamycin (mTOR) by the PI3K/Akt signaling pathway is recognized as a key growth signaling factor which integrates various growth signals in cells. mTOR contributes to the increase of muscle mass by inducing muscle protein synthesis through activation of two factors that initiate mRNA translation, i.e., 4E-binding protein (4EBP1) and phosphorylated 70-kDa ribosomal S6 kinase (p70S6K) (The Korea Journal of Sports Science, 20(3): 1551-1561, 2011; The International Journal of Biochemistry and Cell Biology, 43(9): 1267-1276, 2011). On the contrary, when forkhead box (FoxO), which is a transcription factor, moves from the cytoplasm into the nucleus, the expression of atrogin-1 and MuRF1, which are E3 ubiquitin ligase factors involved in protein degradation, is increased (Disease Models and Mechanisms, 6: 25-39, 2013). If their expression level increases, proteolysis in muscle is accelerated and, thus, muscle mass is decreased. Therefore, acceleration of mTOR activation and inhibition of atrogin-1 and MuRF1 expression lead to increased amount of muscle proteins and result in increased muscle mass.

Muscle cell differentiation and muscle formation are regulated by various muscle regulatory factors. Among the factors, MyoD is the most important factor in the fusion of myoblasts. The muscle fibers formed through this process form a bundle and eventually form muscles (Cellular and Molecular Life Sciences, 70: 4117-4130, 2013).

Cudrania tricuspidata has been traditionally used for treatment of eczema, phthisis, chronic back pain, bruise, etc. and is used as a folk medicine for treatment of such diseases as palsy, etc. (Korean Journal of Food Science and Technology, 33(1): 128-134, 2001). The skin-whitening (Korean Chemical Engineering Research, 52(6): 701-705, 2014), antioxidant (Korean Journal of Medicinal Crop Science, 17(2): 115-120, 2009), antibacterial (Korean Journal of Medicinal Crop Science, 17(5): 335-340, 2009) and anti-obesity (Nutrients 7(12), 10480-10490, 2015) activities of Cudrania tricuspidata have been reported thus far.

However, the prevention and treatment of a muscular disease, or improvement of muscular function by Cudrania tricuspidata, a pulverization product of Cudrania tricuspidata or a Cudrania tricuspidata extract has not been known yet.

### DISCLOSURE

### Technical Problem

The inventors of the present disclosure have searched for natural substances that have superior activity of regulating muscular function and can be used safely. As a result, they have identified that Cudrania tricuspidata such as a pulverization product of Cudrania tricuspidata or a Cudrania tricuspidata extract has the activity of alleviating, treating or improving a muscular disease, or improving muscular function, and have completed the present disclosure.

Accordingly, the present disclosure is directed to providing a food composition for alleviating or preventing a muscular disease, or improving muscular function, which contains Cudrania tricuspidata such as a pulverization product of Cudrania tricuspidata or a Cudrania tricuspidata extract as an active ingredient.

The present disclosure is also directed to providing a pharmaceutical composition for treating or preventing a muscular disease, which contains Cudrania tricuspidata such as a pulverization product of Cudrania tricuspidata or a Cudrania tricuspidata extract as an active ingredient.

The present disclosure is also directed to providing a cosmetic composition for alleviating or preventing a muscular disease, or improving muscular function, which contains Cudrania tricuspidata such as a pulverization product of Cudrania tricuspidata or a Cudrania tricuspidata extract as an active ingredient.

The present disclosure is also directed to providing a feed additive for alleviating or preventing a muscular disease, or improving muscular function, which contains Cudrania tricuspidata such as a pulverization product of Cudrania tricuspidata or a Cudrania tricuspidata extract as an active ingredient.

### Technical Solution

The present disclosure provides a food composition for alleviating or preventing a muscular disease, or improving muscular function, which contains Cudrania tricuspidata such as a pulverization product of Cudrania tricuspidata or a Cudrania tricuspidata extract as an active ingredient.

The present disclosure also provides a pharmaceutical composition for treating or preventing a muscular disease, which contains Cudrania tricuspidata such as a pulverization product of Cudrania tricuspidata or a Cudrania tricuspidata extract as an active ingredient.

The present disclosure also provides a cosmetic composition for alleviating or preventing a muscular disease, or improving muscular function, which contains Cudrania tricuspidata such as a pulverization product of Cudrania tricuspidata or a Cudrania tricuspidata extract as an active ingredient.

The present disclosure also provides a feed additive for alleviating or preventing a muscular disease, or improving muscular function, which contains Cudrania tricuspidata such as a pulverization product of Cudrania tricuspidata or a Cudrania tricuspidata extract as an active ingredient.

The present disclosure also provides a method for treating a muscular disease by administering the above-described composition to human or a non-human animal.

The present disclosure also provides a novel use of Cudrania tricuspidata such as a pulverization product of Cudrania tricuspidata or a Cudrania tricuspidata extract for preparation of a medicine for treating a muscular disease.

### Advantageous Effects

Cudrania tricuspidata such as a pulverization product of Cudrania tricuspidata or a Cudrania tricuspidata extract according to the present disclosure has a superior effect of increasing the activity of mTOR, which is involved in muscle protein synthesis, and inhibiting the mRNA expression of MuRF1 and atrogin-1, which are involved in muscle protein degradation. In addition, it may prevent, treat or alleviate the decline in muscular function, muscle loss, etc. caused by various diseases by increasing muscle mass and improving muscular function. Since the Cudrania tricuspidata of the present disclosure is a natural substance, it can be used for medicines, foods or cosmetics safely without side effects.

### BRIEF DESCRIPTION OF DRAWINGS

FIG. 1 shows a result of investigating the increase of mTOR activity in L6 muscle cells depending on treatment with a Cudrania tricuspidata leaf ethanol extract.

FIG. 2 shows a result of investigating the decrease of the mRNA expression level of atrogin-1 and MuRF1 in L6 muscle cells depending on treatment with a Cudrania tricuspidata leaf ethanol extract.

FIG. 3 shows a result of investigating the decrease of the mRNA expression level of atrogin-1 and MuRF1 in L6 muscle cells depending on treatment with a Cudrania tricuspidata fruit hot water or ethanol extract.

FIG. 4 shows a result of investigating the decrease of the mRNA expression level of atrogin-1 and MuRF1 in L6 muscle cells depending on treatment with a Cudrania tricuspidata branch hot water or ethanol extract.

FIG. 5 shows a result of investigating the improvement of muscle strength in muscle atrophy-induced mouse depending on treatment with a Cudrania tricuspidata leaf 50% ethanol extract.

FIG. 6 shows a result of investigating the increase of muscle volume in muscle atrophy-induced mouse depending on treatment with a Cudrania tricuspidata leaf 50% ethanol extract.

FIG. 7 shows a result of investigating the increase of tibialis anterior muscle weight in muscle atrophy-induced mouse depending on treatment with a Cudrania tricuspidata leaf 50% ethanol extract.

### BEST MODE

Hereinafter, the present disclosure is described specifically.

The present disclosure provides a food composition for alleviating or preventing a muscular disease, or improving muscular function, which contains Cudrania tricuspidata such as a pulverization product of Cudrania tricuspidata or a Cudrania tricuspidata extract; a pharmaceutical composition for treating or preventing a muscular disease, which contains Cudrania tricuspidata such as a pulverization product of Cudrania tricuspidata or a Cudrania tricuspidata extract; a cosmetic composition for alleviating or preventing a muscular disease, or improving muscular function, which contains Cudrania tricuspidata such as a pulverization product of Cudrania tricuspidata or a Cudrania tricuspidata extract; a feed additive for alleviating or preventing a muscular disease, or improving muscular function, which contains Cudrania tricuspidata such as a pulverization product of Cudrania tricuspidata or a Cudrania tricuspidata extract; or a method for treating a muscular disease, which includes applying Cudrania tricuspidata such as a pulverization product of Cudrania tricuspidata or a Cudrania tricuspidata extract to human or a non-human mammal.

In the present disclosure, the ‘Cudrania tricuspidata’ refers to the leaf, fruit, branch or a combination thereof of Cudrania tricuspidata, which belongs to the family Moraceae, and includes a pulverization product of Cudrania tricuspidata or a Cudrania tricuspidata extract.

In the present disclosure, the ‘pulverization product of Cudrania tricuspidata’ may be prepared from the dried leaf, fruit or branch of Cudrania tricuspidata according to a method that can be easily carried out by those having ordinary knowledge in the art to which the present disclosure belongs into a form that can be easily ingested by a mammal including human and the active ingredient of which can be easily released from the dried pulverization product in the intestine after the ingestion and can be easily absorbed into the body of the mammal. In this regard, although the shape or size of the dried pulverization product particles is not limited, they may be specifically prepared into fine powder if possible to ensure easy release of the active ingredient. In an exemplary embodiment of the present disclosure, the pulverization product is prepared by pulverizing the dried Cudrania tricuspidata leaf, fruit or branch with a mixer.

In the present disclosure, the ‘Cudrania tricuspidata extract’ refers to one obtained by extracting the leaf, fruit or branch of Cudrania tricuspidata with a suitable solvent, and includes all of an extract, a diluted or concentrated solution of the extract, a dried product obtained by drying the extract, or a crude purification product or purification product thereof. The Cudrania tricuspidata extract may be obtained by extracting Cudrania tricuspidata with one or more solvent selected from a group consisting of water, a C₁₋₆ organic solvent and a subcritical or supercritical fluid, although not being limited thereto.

The Cudrania tricuspidata extract of the present disclosure may be prepared by a common extraction method in the art such as hot extraction, cold extraction, ultrasonic extraction, filtration, reflux extraction, etc., and the Cudrania tricuspidata may be one which has been purchased commercially, harvested from the nature or cultivated.

The Cudrania tricuspidata extract according to the present disclosure may be isolated according to a common method of preparing an extract from a natural substance known in the art, i.e., using a common solvent under common conditions of temperature and pressure.

The term “fraction” used in present disclosure refers to a product obtained by a fractionation method of isolating a specific ingredient or a group of ingredients from a mixture containing various components. In the present disclosure, the term means a product obtained by a fractionation method of isolating a specific component or a group of components from the prepared Cudrania tricuspidata extract.

In order to obtain the Cudrania tricuspidata fraction according to the present disclosure, a common fractionation solvent known in the art, for example, a polar solvent such as water, a C₁₋₄ anhydrous or hydrated lower alcohol such as ethanol and methanol, etc., a nonpolar solvent such as hexane, butanol, ethyl acetate, chloroform, dichloromethane, etc., or a mixture solvent thereof may be used, although not being limited thereto.

The Cudrania tricuspidata fraction of the present disclosure may include one obtained by further purifying the fraction. For example, the Cudrania tricuspidata extract according to the present disclosure includes fractions obtained by passing through an ultrafiltration membrane having a predetermined molecular weight cut-off and fractions obtained through various further purification methods such as separation by various chromatography techniques (based on size, charge, hydrophobicity or affinity).

In the present disclosure, ‘muscle’ collectively refers to tendon, muscle and sinew, and ‘muscular function’ refers to the ability of muscle to exert force through muscle contraction, and encompasses muscle strength which is the ability of muscle to exert maximum contraction to overcome resistance, muscular endurance which is the ability of muscle to repeat contraction and relaxation for a given weight in terms of the duration or number of the repetitions, and muscle power which is the ability of muscle to exert strong power in a short period of time. The term ‘improvement of muscular function’ in the present disclosure refers to making muscular function better by increasing muscle mass.

The present disclosure provides a pharmaceutical composition for preventing or treating a muscular disease, or improving muscular function, a food composition for preventing or alleviating a muscular disease, or improving muscular function, or a cosmetic composition for preventing or alleviating a muscular disease, or improving muscular function, which contains Cudrania tricuspidata, a pulverization product of Cudrania tricuspidata or a Cudrania tricuspidata extract as an active ingredient. In a specific exemplary embodiment, the Cudrania tricuspidata extract may be a hot water extract, an ethanol extract, an ethyl acetate extract, a hexane extract, an ultrahigh pressure extract, a subcritical extract or a supercritical extract, although not being limited thereto.

In a specific exemplary embodiment, the Cudrania tricuspidata extract may be obtained by extracting the leaf, fruit or branch of Cudrania tricuspidata with one or more solvent selected from a group consisting of water, a C₁₋₆ organic solvent and a subcritical or supercritical fluid. It may be also be obtained by extracting Cudrania tricuspidata under an ultrahigh pressure condition of 100 MPa or higher. If necessary, it may be prepared by further including filtration and concentration steps according to the methods known in the art.

In a specific exemplary embodiment, the C₁₋₆ organic solvent may be one or more selected from a group consisting of a C₁₋₆ alcohol, acetone, ether, benzene, chloroform, ethyl acetate, methylene chloride, hexane, cyclohexane and petroleum ether.

As a specific exemplary embodiment of the present disclosure, the inventors of the present disclosure have prepared a Cudrania tricuspidata extract by extracting a pulverization product of the dried leaf, fruit or branch of Cudrania tricuspidata repeatedly at room temperature using ethanol, ethyl acetate or hexane as a solvent or through hot water extraction, ultrahigh pressure extraction or subcritical fluid extraction.

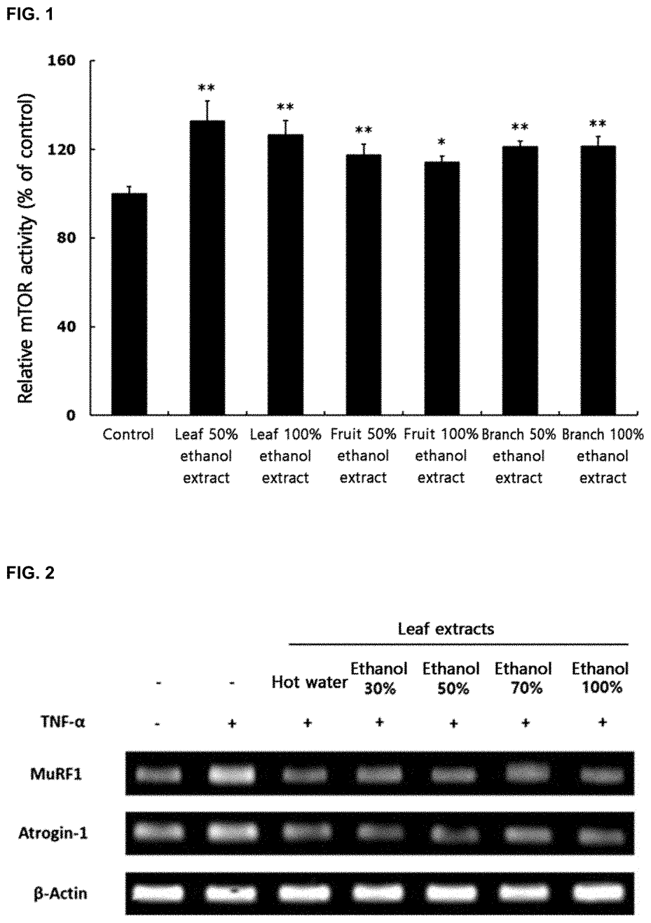

When the prepared 50% and 100% ethanol extracts of the leaf, fruit or branch of Cudrania tricuspidata were treated to L6 muscle cells and the activity in the muscle cells was investigated, it was confirmed that the protein expression of p-mTOR involved in muscle protein synthesis was increased significantly (FIG. 1). Additionally, it was confirmed that the ethyl acetate extract, hexane extract, ultrahigh pressure extract, subcritical extract and supercritical extract of Cudrania tricuspidata also significantly increase the protein expression of p-mTOR in L6 muscle cells (Table 1). In addition, when the hot water and ethanol extracts of the leaf, fruit or branch of Cudrania tricuspidata were treated to L6 muscle cells and the activity in the muscle cells was investigated, it was confirmed that the mRNA expression of MuRF-1 and atrogin-1 involved in muscle protein degradation is inhibited (FIGS. 2-4).

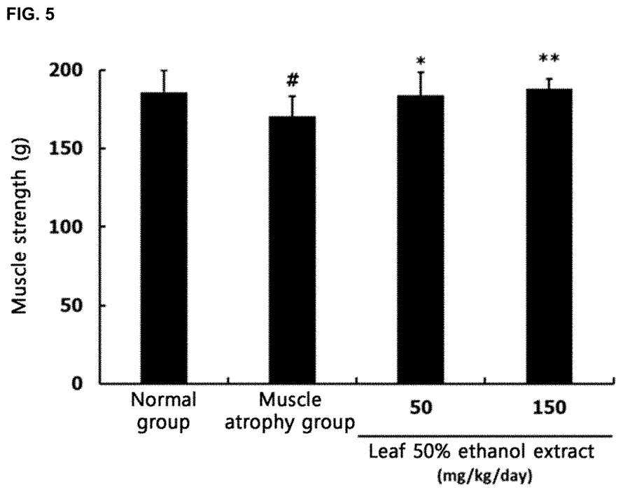

When a Cudrania tricuspidata leaf 50% ethanol was treated to an immobilized mouse model using a skin stapler, muscle strength, muscle volume and muscle weight were increased significantly, respectively, as compared to a control group (FIGS. 5-7). Furthermore, it was confirmed that all of the Cudrania tricuspidata leaf pulverization product, leaf hot water extract, fruit pulverization product, fruit hot water extract, branch pulverization product and branch hot water extract resulted in significant increase in muscle strength and muscle weight as compared to the control group (Table 2).

When the composition for preventing and treating a muscular disease of the present disclosure is a pharmaceutical composition, it may be used to prevent or treat a muscular disease caused by muscular wasting or degeneration. Muscular wasting and degeneration are caused by genetic factors, acquired factors, aging, etc. Muscular wasting is characterized by gradual loss of muscle mass and weakening and degeneration of muscle, particularly skeletal muscle or voluntary muscle and cardiac muscle. Diseases related with the muscular wasting and degeneration may include atony, muscular atrophy, muscular dystrophy, muscle degeneration, muscle weakness, cachexia, sarcopenia, etc. The composition of the present disclosure has an effect of increasing muscle mass, and the muscle is not limited in type.

The pharmaceutical composition for preventing or treating a muscular disease, or improving muscular function of the present disclosure may contain a pharmaceutically acceptable carrier.

The pharmaceutically acceptable carrier may include, for example, a carrier for oral administration or a carrier for parenteral administration. The carrier for oral administration may include lactose, starch, a cellulose derivative, magnesium stearate, stearic acid, etc. And, the carrier for parenteral administration may include water, a suitable oil, saline, water-soluble glucose, glycol, etc. In addition, a stabilizer or a preservative may be further contained. Suitable stabilizers include antioxidants such as sodium hydrogen sulfite, sodium sulfite or ascorbic acid. Suitable preservatives include benzalkonium chloride, methyl- or propylparaben and chlorobutanol. Other pharmaceutically acceptable carriers may be found among those listed in the following literature: Remington's Pharmaceutical Sciences, 19th ed., Mack Publishing Company, Easton, Pa., 1995.

The pharmaceutical composition of the present disclosure may be administered to mammals including human by any method. For example, it may be administered orally or parenterally. Parenteral administration methods include, but are not limited to, intravenous, intramuscular, intraarterial, intramedullary, intradural, intracardiac, transdermal, subcutaneous, intraperitoneal, intranasal, enteral, topical, sublingual or rectal administration.

The pharmaceutical composition of the present disclosure may be formulated into a preparation for oral or parenteral administration depending on the route of administration as described above. It may be formulated using one or more buffers (e.g., saline or PBS), antioxidants, bacteristats, chelating agents (e.g., EDTA or glutathione), fillers, extenders, binder, adjuvants (e.g., aluminum hydroxide), suspending agents, thickeners, wetting agents, disintegrants, surfactants, diluents or excipients.

Solid preparations for oral administration include a tablet, a pill, a powder, a granule, a liquid, a gel, a syrup, a slurry, a suspension, a capsule, etc. These solid preparations may be prepared by mixing at least one excipient, for example, starch (corn starch, wheat starch, rice starch, potato starch, etc.), calcium carbonate, sucrose, lactose, dextrose, sorbitol, mannitol, xylitol, erythritol, maltitol, cellulose, methyl cellulose, sodium carboxymethyl cellulose, hydroxypropylmethyl cellulose, gelatin, etc. in the pharmaceutical composition of the present disclosure. For example, a tablet or a sugar-coated tablet may be obtained by mixing the active ingredient with a solid excipient, pulverizing the mixture, adding a suitable adjuvant and then processing into a granule mixture.

In addition to a simple excipient, a lubricant such as magnesium stearate and talc is also used. Liquid preparations for oral administration, which include a suspension, a liquid for internal use, an emulsion, a syrup, etc., may contain, in addition to a commonly used simple diluent such as water or liquid paraffin, various excipients such as a wetting agent, a sweetener, an aromatic, a preservative, etc.

In some cases, crosslinked polyvinylpyrrolidone, agar, alginic acid, sodium alginate, etc. may be added as a disintegrant, and an anticoagulant, a lubricant, a wetting agent, a flavorant, an emulsifier, an antiseptic, etc. may be further included.

For parenteral administration, the pharmaceutical composition of the present disclosure may be formulated, along with a suitable parenteral carrier, in the form of an injection, a percutaneous preparation or a nasal inhaler according to methods known in the art. The injection should be sterilized and protected against contamination by microorganisms such as bacteria and fungi. Examples of a carrier suitable for the injection may include a solvent or a dispersion medium including water, ethanol, a polyol (e.g., glycerol, propylene glycol, liquid polyethylene glycol, etc.), a mixture thereof and/or vegetable oil, although not being limited thereto. More specifically, an isotonic solution such as Hank's solution, Ringer's solution, triethanolamine-containing phosphate-buffered saline (PBS), sterilized water for injection, 10% ethanol, 40% propylene glycol and 5% dextrose may be used as a suitable carrier. Also, various antibacterial agents and antifungal agents such as paraben, chlorobutanol, phenol, sorbic acid, thimerosal, etc. may be additionally used to protect the injection against contamination by microorganisms. Also, in most cases, the injection may further include an isotonic agent such as a sugar or sodium chloride.

The percutaneous preparation includes an ointment, a cream, a lotion, a gel, a liquid for external use, a paste, a liniment, an aerosol, etc. Here, ‘percutaneous administration’ refers to administration of a pharmaceutical composition topically to skin so that an effective amount of the active ingredient contained in the pharmaceutical composition is delivered into the skin.

In the case of the inhaler, the compound used according to the present disclosure may be delivered conveniently in the form of a pressurized pack or an aerosol spray from a nebulizer using a suitable propellant, e.g., dichlorofluoromethane, trichlorofluoromethane, dichlorotetrafluoroethane, carbon dioxide or other suitable gases. In the case of a pressurized aerosol, the unit dosage may be determined by providing a valve that delivers a metered amount. For example, a gelatin capsule and a cartridge used in an inhaler or an insufflator may be formulated to contain a powder mixture of a compound and a suitable powder base such as lactose or starch. Formulations for parenteral administration are listed in Remington's Pharmaceutical Science, 15th Edition, 1975. Mack Publishing Company, Easton, Pa. 18042, Chapter 87: Blaug, Seymour, which is a prescription manual commonly known in pharmaceutical chemistry.

The pharmaceutical composition of the present disclosure for preventing or treating a muscular disease, or improving muscular function may provide a preferred effect of preventing or treating a muscular disease, or improving muscular function when containing an effective amount of a Cudrania tricuspidata extract. In the present disclosure, the ‘effective amount’ refers to an amount that results in better response than that of a negative control group, and specifically refers to an amount sufficient to improve muscular function. The pharmaceutical composition of the present disclosure may contain 0.01-99.99% of the Cudrania tricuspidata extract and a pharmaceutically acceptable carrier as the remainder. The effective amount of the Cudrania tricuspidata extract contained in the pharmaceutical composition of the present disclosure will vary depending on the type of the composition.

The total effective amount of the pharmaceutical composition of the present disclosure may be administered to a patient as a single dose or a multiple dose according to a long-term fractionated treatment protocol. The content of the active ingredient in the pharmaceutical composition of the present disclosure may vary depending on the severity of a disease. In the case of parenteral administration, the pharmaceutical composition may be administered as a single dose or a multiple dose so that specifically 0.01-50 mg, more specifically 0.1-30 mg, of the Cudrania tricuspidata extract is administered per 1 kg body weight a day. And, in the case of oral administration, it may be administered as a single dose or a multiple dose so that specifically 0.01-100 mg, more specifically 0.01-10 mg, of the Cudrania tricuspidata extract is administered per 1 kg body weight a day. However, since the effective administration dose of the Cudrania tricuspidata extract is determined in consideration of various factors such as not only the administration route of the pharmaceutical composition and the frequency of treatment, but also the age, body weight, health status and sex of a patient, the severity of a disease, diet, excretion rate, etc., those having ordinary knowledge in the art may be able to determine a suitable effective dose of the Cudrania tricuspidata extract depending on particular use for prevention or treatment of a muscular disease. The pharmaceutical composition according to the present disclosure is not particularly limited to a specific formulation, administration route and administration method as long as the effect of the present disclosure is attained.

The pharmaceutical composition for preventing and treating a muscular disease, or improving muscular function of the present disclosure may be used either alone or in combination with a method that involves surgery, radiation therapy, hormone therapy, chemotherapy or use of a biological response modifier.

The pharmaceutical composition for preventing and treating a muscular disease, or improving muscular function of the present disclosure may also be provided as a formulation for external use that contains the Cudrania tricuspidata extract as an active ingredient.

When the pharmaceutical composition for preventing and treating a muscular disease, or improving muscular function of the present disclosure is used as a formulation for external use on skin, it may further contain an adjuvant commonly used in the field of dermatology, such as a fatty substance, an organic solvent, a solubilizer, a thickener, a gelling agent, a softening agent, an antioxidant, a suspending agent, a stabilizer, a foaming agent, an aromatic, a surfactant, water, an ionic emulsifier, a nonionic emulsifier, a filler, a sequestering agent, a chelating agent, a preservative, a vitamin, a blocking agent, a wetting agent, an essential oil, a dye, a pigment, a hydrophilic activator, a lipophilic activator, or any other ingredient commonly used in a preparation for external use on skin such as a lipid vesicle, etc. In addition, the ingredients may be contained in an amount commonly used in the field of dermatology.

When the pharmaceutical composition for preventing and treating a muscular disease, or improving muscular function of the present disclosure is provided as a preparation for external use on skin, it may be in the form of an ointment, a patch, a gel, a cream, a spray, etc., although not being limited thereto.

The composition for preventing and alleviating a muscular disease, or improving muscular function of the present disclosure may also be a food composition. When the composition for preventing and alleviating a muscular disease, or improving muscular function of the present disclosure is a food composition, it may be used to prevent or alleviate a muscular disease caused by muscular wasting or degeneration. Muscular wasting and degeneration are caused by genetic factors, acquired factors, aging, etc. Muscular wasting is characterized by gradual loss of muscle mass and weakening and degeneration of muscle, especially skeletal muscle or voluntary muscle or cardiac muscle. Examples of related diseases include atony, muscular atrophy, muscular dystrophy, muscle degeneration, muscle weakness, cachexia, sarcopenia, etc. The composition of the present disclosure is effective for increasing muscle mass, and the type of muscle affected by the composition is not limited.

The food composition of the present disclosure includes all types such as a functional food, a nutritional supplement, a health food, a food additive, an animal feed, etc., and is targeted for feeding animals including human and livestock. The food composition may be prepared into various forms according to a common method known in the art.

General foods may be prepared by adding Cudrania tricuspidata, a pulverization product of Cudrania tricuspidata or a Cudrania tricuspidata extract to beverages (including alcoholic beverages), fruit or processed foods thereof (e.g., canned fruit, bottled fruit, jam, marmalade, et.), fish, meat or processed foods thereof (e.g., ham, sausage, corned beef, etc.), bread or noodles (e.g., udon, buckwheat noodles, ramen, spaghetti, macaroni, etc.), fruit juices, drinks, cookies, taffies, dairy products (e.g., butter, cheese, etc.), edible vegetable oils, margarine, vegetable proteins, retort foods, frozen foods, seasonings (e.g., soybean paste, soy sauce, other sauces, etc.), or the like, although not being limited thereto. And, a nutritional supplement may be prepared by adding Cudrania tricuspidata, a pulverization product of Cudrania tricuspidata or a Cudrania tricuspidata extract to a capsule, a tablet, a pill, etc., although not being limited thereto. Also, a functional health food may be prepared by liquefying, granulating, encapsulating or powdering Cudrania tricuspidata, a pulverization product of Cudrania tricuspidata or a Cudrania tricuspidata extract itself, for example, in the form of tea, juice or a drink for drinking. In addition, Cudrania tricuspidata, a pulverization product of Cudrania tricuspidata or a Cudrania tricuspidata extract may be processed into a powder or a concentrate for use as a food additive. In addition, Cudrania tricuspidata, a pulverization product of Cudrania tricuspidata or a Cudrania tricuspidata extract may be mixed together with an active ingredient known to be effective in preventing and alleviating a muscular disease, or improving muscular function to prepare a composition.

When the composition for preventing and alleviating a muscular disease, or improving muscular function of the present disclosure is used as a health beverage composition, the health beverage composition may further contain various flavorants, natural carbohydrates, etc. as ordinary beverages. The natural carbohydrate may be a monosaccharide such as glucose or fructose; a disaccharide such as maltose or sucrose; a polysaccharide such as dextrin or cyclodextrin; or a sugar alcohol such as xylitol, sorbitol, erythritol, etc. As a sweetener, a natural sweetener such as thaumatin or stevia extract; or a synthetic sweetener such as saccharin or aspartame may be used. The concentration of the natural carbohydrate is generally about 0.01-0.04 g, specifically about 0.02-0.03 g, per 100 mL of the composition of the present disclosure.

Cudrania tricuspidata, a pulverization product of Cudrania tricuspidata or a Cudrania tricuspidata extract may be contained in a food composition for preventing or alleviating a muscular disease, or improving muscular function as an active ingredient. In this case, its amount may be an amount effective for accomplishing the prevention and alleviation of a muscular disease, or improvement of muscular function. Specifically, the amount may be 0.01-100 wt % based on the total weight of the composition, although not being specially limited thereto. The food composition of the present disclosure may be prepared by mixing Cudrania tricuspidata, a pulverization product of Cudrania tricuspidata or a Cudrania tricuspidata extract with another active ingredient known to be effective in preventing and alleviating a muscular disease, or improving muscular function.

In addition to the above, the health food of the present disclosure may contain various nutrients, vitamins, electrolytes, flavoring agents, colorants, pectic acid, salts of pectic acid, alginic acid, salts of alginic acid, organic acids, protective colloidal thickeners, pH-adjusting agents, stabilizers, antiseptics, glycerin, alcohols, carbonating agents, etc. In addition, the health food of the present disclosure may further contain fruit flesh for preparing a natural fruit juice, a fruit juice beverage or a vegetable beverage. These ingredients may be used either alone or in combination. Although the concentration of these additives is of no great importance, it is generally selected within a range of 0.01-0.1 part by weight based on 100 parts by weight of the composition of the present disclosure.

The composition for preventing and alleviating a muscular disease, or improving muscular function of the present disclosure may also be a cosmetic composition. The cosmetic composition of the present disclosure may be prepared into a basic cosmetic composition (lotion, cream, essence, facial cleanser such as cleansing foam and cleansing water, pack or body oil), a color cosmetic composition (foundation, lipstick, mascara or makeup base), a hair product composition (shampoo, rinse, hair conditioner or hair gel), a soap, etc. using Cudrania tricuspidata, a pulverization product of Cudrania tricuspidata or a Cudrania tricuspidata extract as an active ingredient together with a dermatologically acceptable excipient.

The excipient may include, for example, a skin softener, a skin penetration enhancer, a colorant, an aromatic, an emulsifier, a thickener and a solvent, although not being limited thereto. In addition, a flavorant, a colorant, a sterilizer, an antioxidant, an antiseptic, a moisturizer, etc., may be further contained, and a thickener, a mineral, a synthetic polymer, etc. may be contained for improvement of physical properties. For example, when the cosmetic composition of the present disclosure is prepared into a facial cleanser or a soap, it may be prepared easily by adding Cudrania tricuspidata, a pulverization product of Cudrania tricuspidata or a Cudrania tricuspidata extract to a common facial cleanser or soap base. A cream may be prepared by adding Cudrania tricuspidata, a pulverization product of Cudrania tricuspidata or a Cudrania tricuspidata extract to a common oil-in-water (O/W) cream base. In addition, a flavorant, a chelating agent, a colorant, an antioxidant, an antiseptic, etc. and a synthetic or natural material for prevention of physical properties, such as a protein, a mineral, a vitamin, etc., may be further added.

The content of Cudrania tricuspidata, a pulverization product of Cudrania tricuspidata or a Cudrania tricuspidata extract contained in the cosmetic composition of the present disclosure may be specifically 0.001-10 wt %, more specifically 0.01-5 wt %, based on the total weight of the composition, although not being limited thereto. If the content is below 0.001 wt %, the desired effect of preventing and treating a muscular disease, or improving muscular function cannot be expected. And, if the content exceeds 10 wt %, there may be difficulties in terms of safety or formulability.

The composition of the present disclosure may be added to an animal feed additive or an animal feed composition for the purpose of preventing or alleviating a muscular disease.

In the present disclosure, the term “animal feed additive” includes a substance added to an animal feed for various purposes, such as supplementation of nutrients, prevention of body weight reduction, enhancement of the availability of cellulose in an animal feed, improvement of oiliness, prevention of reproductive failure, improvement of pregnancy rate, prevention of heat stress during summer, etc. The animal feed additive of the present disclosure corresponds to the complementary animal feed under the Control of Livestock and Fish Feed Act, and may further contain a mineral such as sodium bicarbonate, bentonite, magnesium oxide, complex mineral, etc., a trace mineral such as zinc, copper, cobalt, selenium, etc., a vitamin such as carotene, vitamin A D and E, nicotinic acid, vitamin B complex, etc., a protected amino acid such as methionine, lysine, etc., a protected fatty acid such as a fatty acid calcium salt, etc., a probiotic (lactic acid bacteria product) such as yeast culture, fermented fungi, etc.

In the present disclosure, the term “animal feed” refers to any natural or artificial diet, meal, etc. that can be eaten, taken or digested by animals. An animal feed containing the composition for preventing or alleviating a muscular disease according to the present disclosure as an active ingredient may be prepared into various forms known in the art, specifically including an enriched animal feed, a bulky animal feed and/or a special animal feed, although not being limited thereto.

The enriched animal feed includes seeds or fruits including grains such as wheat, oats, corn, etc., brans obtained as byproducts after polishing grains, including rice bran, wheat bran, barley bran, etc., seedcakes obtained as byproducts after extracting oil from bean, rapeseed, sesame, linseed, coconut palm, etc., starch dregs obtained after extracting starch from sweet potato, potato, etc., fish solubles obtained by concentrating liquid obtained from fish meals or fish dregs, meat meals, blood meals, feather meals, dry whey obtained by drying whey obtained during preparation of cheese from skim milk or milk and casein from skim milk, yeasts, chlorella and seaweeds, although not being limited thereto.

The bulky animal feed includes green forage feed such as wild grass, grass, zero-grazed crop, etc., root vegetables such as feed turnip, feed beet, rutabaga which is a type of turnip, etc., silage prepared through lactic acid fermentation of green forage, zero-grazed crop, crops, etc. in a silo, hay prepared by drying wild grass or grass, straw of crops for breeding stock, and leaves of leguminous plants, although not being limited thereto. The special animal feed includes mineral feed such as oyster shell, rock salt, etc., urea feed such as urea or a derivative thereof such as diureidoisobutane, etc., an animal feed additive or a dietary supplement added in a trace amount to assorted feed to as a supplement for a lacking ingredient or to improve storage characteristics, although not being limited thereto.

The feed additive for preventing or alleviating a muscular disease according to the present disclosure may be prepared by adding an effective amount of Cudrania tricuspidata, a pulverization product of Cudrania tricuspidata or a Cudrania tricuspidata extract according to various animal feed preparation methods known in the art.

The feed additive according to the present disclosure may be used without limitation to any subject for the purpose of preventing or alleviating a muscular disease. For example, it may be applied to any subject including non-human mammals such as cow, horse, pig, goat, sheep, dog, cat, rabbit, etc., birds, fish, or the like.

Hereinafter, the present disclosure is described in more detail through examples.

However, the following examples only illustrate the present disclosure and the contents pf the present disclosure are not limited by the examples.

### [Example 1] Preparation of Cudrania tricuspidata Leaf Extract

<1-1> Preparation of Cudrania tricuspidata Leaf Hot Water Extract

After pulverizing dried Cudrania tricuspidata leaf with a mixer, 10 g of the pulverized Cudrania tricuspidata leaf sample was added to 100 mL of water and extracted for 3 hours at 80° C. The extracted sample was filtered through Whatman No. 1 filter paper under reduced pressure, and a Cudrania tricuspidata leaf hot water extract was obtained by removing the solvent from the filtered extract by concentrating with a rotary vacuum evaporator.

<1-2> Preparation of Cudrania tricuspidata Leaf 30% Ethanol Extract

After pulverizing dried Cudrania tricuspidata leaf with a mixer, 10 g of the pulverized Cudrania tricuspidata leaf sample was added to 100 mL of 30% ethanol and extracted for 3 hours at 40° C. The extracted sample was filtered through Whatman No. 1 filter paper under reduced pressure, and a Cudrania tricuspidata leaf 30% ethanol extract was obtained by removing the solvent from the filtered extract by concentrating with a rotary vacuum evaporator. The 30% ethanol extract means an extract extracted using a mixture solvent of 70 wt % of water and 30 wt % of ethanol.

<1-3> Preparation of Cudrania tricuspidata Leaf 50% Ethanol Extract

After pulverizing dried Cudrania tricuspidata leaf with a mixer, 10 g of the pulverized Cudrania tricuspidata leaf sample was added to 100 mL of 50% ethanol and extracted for 3 hours at 40° C. The extracted sample was filtered through Whatman No. 1 filter paper under reduced pressure, and a Cudrania tricuspidata leaf 50% ethanol extract was obtained by removing the solvent from the filtered extract by concentrating with a rotary vacuum evaporator.

<1-4> Preparation of Cudrania tricuspidata leaf 70% ethanol extract

After pulverizing dried Cudrania tricuspidata leaf with a mixer, 10 g of the pulverized Cudrania tricuspidata leaf sample was added to 100 mL of 70% ethanol and extracted for 3 hours at 40° C. The extracted sample was filtered through Whatman No. 1 filter paper under reduced pressure, and a Cudrania tricuspidata leaf 70% ethanol extract was obtained by removing the solvent from the filtered extract by concentrating with a rotary vacuum evaporator.

<1-5> Preparation of Cudrania tricuspidata Leaf 100% Ethanol Extract

After pulverizing dried Cudrania tricuspidata leaf with a mixer, 10 g of the pulverized Cudrania tricuspidata leaf sample was added to 100 mL of 100% ethanol and extracted for 3 hours at 40° C. The extracted sample was filtered through Whatman No. 1 filter paper under reduced pressure, and a Cudrania tricuspidata leaf 100% ethanol extract was obtained by removing the solvent from the filtered extract by concentrating with a rotary vacuum evaporator.

<1-6> Preparation of Cudrania tricuspidata Leaf Ethyl Acetate Extract

After pulverizing dried Cudrania tricuspidata leaf with a mixer, 10 g of the pulverized Cudrania tricuspidata leaf sample was added to 100 mL of ethyl acetate and extracted for 3 hours at 40° C. The extracted sample was filtered through Whatman No. 1 filter paper under reduced pressure, and a Cudrania tricuspidata leaf ethyl acetate extract was obtained by removing the solvent from the filtered extract by concentrating with a rotary vacuum evaporator.

<1-7> Preparation of Cudrania tricuspidata Leaf Hexane Extract

After pulverizing dried Cudrania tricuspidata leaf with a mixer, 10 g of the pulverized Cudrania tricuspidata leaf sample was added to 100 mL of hexane and extracted for 3 hours at 40° C. The extracted sample was filtered through Whatman No. 1 filter paper under reduced pressure, and a Cudrania tricuspidata leaf hexane extract was obtained by removing the solvent from the filtered extract by concentrating with a rotary vacuum evaporator.

<1-8> Preparation of Cudrania tricuspidata Leaf Ultrahigh Pressure Extract

After pulverizing dried Cudrania tricuspidata leaf with a mixer, 1 g of the pulverized Cudrania tricuspidata leaf and 76 mL of 18% ethanol were put in a polyethylene pack and then extracted using an ultrahigh pressure extractor (Frescal MFP-7000; Mitsubishi Heavy Industries) after sealing. The ultrahigh pressure extraction condition was as follows: extraction pressure=320 MPa; extraction time=5 minutes. The extracted sample was filtered through Whatman No. 2 filter paper, and a Cudrania tricuspidata leaf ultrahigh pressure extract was obtained by removing the solvent from the filtered extract by concentrating with a rotary vacuum evaporator.

<1-9> Preparation of Cudrania tricuspidata Leaf Subcritical Extract

After pulverizing dried Cudrania tricuspidata leaf with a mixer, 50 g of the pulverized Cudrania tricuspidata leaf and 1 L of water were put in a subcritical water reactor in a subcritical extractor (Biovan, Gyeonggi, Korea). After sealing, the temperature of the reactor was raised to 200° C. and, when the temperature of the reactor reached 200° C., the temperature was maintained for 20 minutes for extraction. 20 minutes later, the extract was transferred to a storage tank to which cooling water was supplied to be rapidly cooled down to 30° C. Then, in order to separate the suspended residue, centrifugation was performed at 3,600 rpm for 30 minutes and only the supernatant was taken. A Cudrania tricuspidata leaf subcritical extract was obtained by completely removing the solvent using a freeze dryer (Ilshin Lab Co. Ltd., Seoul, Korea).

<1-10> Preparation of Cudrania tricuspidata Leaf Supercritical Extract

After pulverizing dried Cudrania tricuspidata leaf with a mixer, 10 g of the pulverized Cudrania tricuspidata leaf was filled in a sample cartridge and extracted using a supercritical fluid extractor (SFX 3560, Isco Inc., Lincoln, Nebr., USA). The supercritical extraction condition was as follows: extraction pressure=300 bar; extraction temperature=50° C.; flow rate of supercritical carbon dioxide=60 mL/min; extraction time=2 hours. After the supercritical fluid extraction was completed, a Cudrania tricuspidata leaf supercritical extract was obtained by lowering the pressure of the extractor.

### [Example 2] Preparation of Cudrania tricuspidata Fruit Extract

<2-1> Preparation of Cudrania tricuspidata Fruit Hot Water Extract

After pulverizing dried Cudrania tricuspidata fruit with a mixer, 10 g of the pulverized Cudrania tricuspidata fruit sample was added to 100 mL of water and extracted for 3 hours at 80° C. The extracted sample was filtered through Whatman No. 1 filter paper under reduced pressure, and a Cudrania tricuspidata fruit hot water extract was obtained by removing the solvent from the filtered extract by concentrating with a rotary vacuum evaporator.

<2-2> Preparation of Cudrania tricuspidata Fruit 30% Ethanol Extract

After pulverizing dried Cudrania tricuspidata fruit with a mixer, 10 g of the pulverized Cudrania tricuspidata fruit sample was added to 100 mL of 30% ethanol and extracted for 3 hours at 40° C. The extracted sample was filtered through Whatman No. 1 filter paper under reduced pressure, and a Cudrania tricuspidata fruit 30% ethanol extract was obtained by removing the solvent from the filtered extract by concentrating with a rotary vacuum evaporator.

<2-3> Preparation of Cudrania tricuspidata Fruit 50% Ethanol Extract

After pulverizing dried Cudrania tricuspidata fruit with a mixer, 10 g of the pulverized Cudrania tricuspidata fruit sample was added to 100 mL of 50% ethanol and extracted for 3 hours at 40° C. The extracted sample was filtered through Whatman No. 1 filter paper under reduced pressure, and a Cudrania tricuspidata fruit 50% ethanol extract was obtained by removing the solvent from the filtered extract by concentrating with a rotary vacuum evaporator.

<2-4> Preparation of Cudrania tricuspidata Fruit 70% Ethanol Extract

After pulverizing dried Cudrania tricuspidata fruit with a mixer, 10 g of the pulverized Cudrania tricuspidata fruit sample was added to 100 mL of 70% ethanol and extracted for 3 hours at 40° C. The extracted sample was filtered through Whatman No. 1 filter paper under reduced pressure, and a Cudrania tricuspidata fruit 70% ethanol extract was obtained by removing the solvent from the filtered extract by concentrating with a rotary vacuum evaporator.

<2-5> Preparation of Cudrania tricuspidata Fruit 100% Ethanol Extract

After pulverizing dried Cudrania tricuspidata fruit with a mixer, 10 g of the pulverized Cudrania tricuspidata fruit sample was added to 100 mL of 100% ethanol and extracted for 3 hours at 40° C. The extracted sample was filtered through Whatman No. 1 filter paper under reduced pressure, and a Cudrania tricuspidata fruit 100% ethanol extract was obtained by removing the solvent from the filtered extract by concentrating with a rotary vacuum evaporator.

<2-6> Preparation of Cudrania tricuspidata Fruit Ultrahigh Pressure Extract

After pulverizing dried Cudrania tricuspidata fruit with a mixer, 1 g of the pulverized Cudrania tricuspidata leaf and 76 mL of 18% ethanol were put in a polyethylene pack and then extracted using an ultrahigh pressure extractor (Frescal MFP-7000; Mitsubishi Heavy Industries) after sealing. The ultrahigh pressure extraction condition was as follows: extraction pressure=320 MPa; extraction time=5 minutes. The extracted sample was filtered through Whatman No. 2 filter paper, and a Cudrania tricuspidata fruit ultrahigh pressure extract was obtained by removing the solvent from the filtered extract by concentrating with a rotary vacuum evaporator.

<2-7> Preparation of Cudrania tricuspidata Fruit Subcritical Extract

After pulverizing dried Cudrania tricuspidata fruit with a mixer, 50 g of the pulverized Cudrania tricuspidata fruit and 1 L of water were put in a subcritical water reactor in a subcritical extractor (Biovan, Gyeonggi, Korea). After sealing, the temperature of the reactor was raised to 200° C. and, when the temperature of the reactor reached 200° C., the temperature was maintained for 20 minutes for extraction. 20 minutes later, the extract was transferred to a storage tank to which cooling water was supplied to be rapidly cooled down to 30° C. Then, in order to separate the suspended residue, centrifugation was performed at 3,600 rpm for 30 minutes and only the supernatant was taken. A Cudrania tricuspidata fruit subcritical extract was obtained by completely removing the solvent using a freeze dryer (Ilshin Lab Co. Ltd., Seoul, Korea).

<2-8> Preparation of Cudrania tricuspidata Fruit Supercritical Extract

After pulverizing dried Cudrania tricuspidata fruit with a mixer, 10 g of the pulverized Cudrania tricuspidata fruit was filled in a sample cartridge and extracted using a supercritical fluid extractor (SFX 3560, Isco Inc., Lincoln, Nebr., USA). The supercritical extraction condition was as follows: extraction pressure=300 bar; extraction temperature=50° C.; flow rate of supercritical carbon dioxide=60 mL/min; extraction time=2 hours. After the supercritical fluid extraction was completed, a Cudrania tricuspidata fruit supercritical extract was obtained by lowering the pressure of the extractor.

### [Example 3] Preparation of Cudrania tricuspidata Branch Extract

<3-1> Preparation of Cudrania tricuspidata Branch Hot Water Extract

After pulverizing dried Cudrania tricuspidata branch with a mixer, 10 g of the pulverized Cudrania tricuspidata branch sample was added to 100 mL of water and extracted for 3 hours at 80° C. The extracted sample was filtered through Whatman No. 1 filter paper under reduced pressure, and a Cudrania tricuspidata branch hot water extract was obtained by removing the solvent from the filtered extract by concentrating with a rotary vacuum evaporator.

<3-2> Preparation of Cudrania tricuspidata Branch 30% Ethanol Extract

After pulverizing dried Cudrania tricuspidata branch with a mixer, 10 g of the pulverized Cudrania tricuspidata branch sample was added to 100 mL of 30% ethanol and extracted for 3 hours at 40° C. The extracted sample was filtered through Whatman No. 1 filter paper under reduced pressure, and a Cudrania tricuspidata branch 30% ethanol extract was obtained by removing the solvent from the filtered extract by concentrating with a rotary vacuum evaporator.

<3-3> Preparation of Cudrania tricuspidata Branch 50% Ethanol Extract

After pulverizing dried Cudrania tricuspidata branch with a mixer, 10 g of the pulverized Cudrania tricuspidata branch sample was added to 100 mL of 50% ethanol and extracted for 3 hours at 40° C. The extracted sample was filtered through Whatman No. 1 filter paper under reduced pressure, and a Cudrania tricuspidata branch 50% ethanol extract was obtained by removing the solvent from the filtered extract by concentrating with a rotary vacuum evaporator.

<3-4> Preparation of Cudrania tricuspidata Branch 70% Ethanol Extract

After pulverizing dried Cudrania tricuspidata branch with a mixer, 10 g of the pulverized Cudrania tricuspidata branch sample was added to 100 mL of 70% ethanol and extracted for 3 hours at 40° C. The extracted sample was filtered through Whatman No. 1 filter paper under reduced pressure, and a Cudrania tricuspidata branch 70% ethanol extract was obtained by removing the solvent from the filtered extract by concentrating with a rotary vacuum evaporator.

<3-5> Preparation of Cudrania tricuspidata Branch 100% Ethanol Extract

After pulverizing dried Cudrania tricuspidata branch with a mixer, 10 g of the pulverized Cudrania tricuspidata branch sample was added to 100 mL of 100% ethanol and extracted for 3 hours at 40° C. The extracted sample was filtered through Whatman No. 1 filter paper under reduced pressure, and a Cudrania tricuspidata branch 100% ethanol extract was obtained by removing the solvent from the filtered extract by concentrating with a rotary vacuum evaporator.

<3-6> Preparation of Cudrania tricuspidata Branch Ultrahigh Pressure Extract

After pulverizing dried Cudrania tricuspidata branch with a mixer, 1 g of the pulverized Cudrania tricuspidata branch and 76 mL of 18% ethanol were put in a polyethylene pack and then extracted using an ultrahigh pressure extractor (Frescal MFP-7000; Mitsubishi Heavy Industries) after sealing. The ultrahigh pressure extraction condition was as follows: extraction pressure=320 MPa; extraction time=5 minutes. The extracted sample was filtered through Whatman No. 2 filter paper, and a Cudrania tricuspidata branch ultrahigh pressure extract was obtained by removing the solvent from the filtered extract by concentrating with a rotary vacuum evaporator.

<3-7> Preparation of Cudrania tricuspidata Branch Subcritical Extract

After pulverizing dried Cudrania tricuspidata branch with a mixer, 50 g of the pulverized Cudrania tricuspidata branch and 1 L of water were put in a subcritical water reactor in a subcritical extractor (Biovan, Gyeonggi, Korea). After sealing, the temperature of the reactor was raised to 200° C. and, when the temperature of the reactor reached 200° C., the temperature was maintained for 20 minutes for extraction. 20 minutes later, the extract was transferred to a storage tank to which cooling water was supplied to be rapidly cooled down to 30° C. Then, in order to separate the suspended residue, centrifugation was performed at 3,600 rpm for 30 minutes and only the supernatant was taken. A Cudrania tricuspidata branch subcritical extract was obtained by completely removing the solvent using a freeze dryer (Ilshin Lab Co. Ltd., Seoul, Korea).

<3-8> Preparation of Cudrania tricuspidata Branch Supercritical Extract

After pulverizing dried Cudrania tricuspidata branch with a mixer, 10 g of the pulverized Cudrania tricuspidata branch was filled in a sample cartridge and extracted using a supercritical fluid extractor (SFX 3560, Isco Inc., Lincoln, Nebr., USA). The supercritical extraction condition was as follows: extraction pressure=300 bar; extraction temperature=50° C.; flow rate of supercritical carbon dioxide=60 mL/min; extraction time=2 hours. After the supercritical fluid extraction was completed, a Cudrania tricuspidata branch supercritical extract was obtained by lowering the pressure of the extractor.

### [Example 4] Muscle Protein Synthesis-Promoting Effect of Cudrania tricuspidata Ethanol Extracts

It is known that the mTOR protein can, when activated through phosphorylation, induce the activation of proteins involved in muscle protein synthesis and muscle mass increase in the PI3K/Akt signaling pathway in muscle cells. Therefore, to confirm the muscle formation-inducing activity of Cudrania tricuspidata, mTOR activity was investigated using an mTOR sandwich ELISA kit (Cell Signaling Technology, Beverly, Mass., USA).

L6 myoblasts (ATCC; Manassas, Va., USA) were seeded onto a 6-well plate with Dulbecco's modified Eagle's medium (DMEM; Hyclone) containing 10% fetal bovine serum (FBS; Hyclone, Logan, Utah, USA) at 1×10⁵ cell/well and then cultured for 24 hours. After the culturing, the L6 cells were differentiated into myotubes by replacing the medium remaining in the well with DMEM (Hyclone) containing 2% horse serum (HS; Hyclone) and culturing further for 6 days. Then, the cells were cultured for 12 hours after treating with each of the Cudrania tricuspidata leaf 50% ethanol extract of Example 1-3, the Cudrania tricuspidata leaf 100% ethanol extract of Example 1-5, the Cudrania tricuspidata fruit 50% ethanol extract of Example 2-3, the Cudrania tricuspidata fruit 100% ethanol extract of Example 2-3, the Cudrania tricuspidata branch 50% ethanol extract of Example 3-3 and the Cudrania tricuspidata branch 100% ethanol extract of Example 3-5 dissolved in DMEM (Hyclone) at 40 μg/mL. After the culturing, the cells were lysed by treating with a cell lysis buffer. Proteins in the obtained cell lysate were quantified to a concentration of 1 mg/mL by the Bradford method (Bio-Rad Laboratories Inc., Hercules, Calif., USA). 50 μL of the cell lysate was dispensed into a microwell attached with an anti-mTOR antibody and incubated at 37° C. for 2 hours. After washing 4 times with a washing buffer and treating with a detection antibody, the cell lysate was incubated at 37° C. for 1 hour. After washing again for a total of 4 times with a washing buffer and adding a horseradish peroxidase-conjugated secondary antibody, the cell lysate was incubated at 37° C. for 30 minutes. Finally, after washing 4 times with a washing buffer and adding a TMB substrate to each well, incubation was conducted at 37° C. for 10 minutes and the TMB reaction was stopped by adding a stop solution. 2 minutes later, the mTOR level in the myotubes treated with the Cudrania tricuspidata leaf extract was measured by measuring absorbance at 450 nm. The experiment was carried out in triplicate and the measurement result was represented as the mean±standard deviation of percentage (%) with respect to a control group. The difference between the groups was analyzed through Duncan's multi-range test by one-way analysis of variance using the SPSS25.0 statistical package (SPSS Inc., Chicago, Ill., USA). A p value smaller than 5% was considered statistically significant.

As shown in FIG. 1, the treatment with the ethanol extracts of the Cudrania tricuspidata leaf, fruit and branch resulted in significantly increased mTOR activity (**p<0.01) as compared to the control group not treated with the extract. This means that the ethanol extracts of the Cudrania tricuspidata leaf, fruit and branch of the present disclosure have superior ability of promoting muscle protein synthesis in muscle cells.

### [Example 5] Muscle Protein Synthesis-Promoting Effect of Cudrania tricuspidata Extracts

mTOR activity was measured in the same manner as in Example 1, except that each of the Cudrania tricuspidata leaf ethyl acetate, hexane, ultrahigh pressure, subcritical and supercritical extracts of Example 1-6 to Example 1-10, the Cudrania tricuspidata fruit subcritical and supercritical extracts of Example 2-6 to Example 2-8, the Cudrania tricuspidata branch subcritical and supercritical extracts of Example 3-6 to Example 3-8 was treated at 40 μg/m L.

As shown in Table 1, the treatment with the Cudrania tricuspidata extracts resulted in significantly increased mTOR activity (**p<0.01) as compared to the control group not treated with the extract. This means that the ethanol extracts of the Cudrania tricuspidata extracts of the present disclosure have superior ability of promoting muscle protein synthesis in muscle cells.

TABLE 1

| Test groups | Relative mTOR activity (%) |
|---|---|
| Example 1-6 | 127.6 ± 2.2** |
| Example 1-7 | 126.1 ± 1.9** |
| Example 1-8 | 131.8 ± 4.0** |
| Example 1-9 | 130.5 ± 1.7** |
| Example 1-10 | 132.6 ± 3.8** |
| Example 2-6 | 122.5 ± 3.9** |
| Example 2-7 | 116.0 ± 2.8** |
| Example 2-8 | 125.5 ± 3.1** |
| Example 3-6 | 126.2 ± 2.6** |
| Example 3-7 | 119.1 ± 3.3** |
| Example 3-8 | 121.9 ± 3.7** |

### [Example 6] Muscle Protein Degradation-Inhibiting Effect of Cudrania tricuspidata Leaf Hot Water and Ethanol Extracts

L6 myoblasts (ATCC), which are muscle cells, were added at 1×10⁵ cell/mL to a 6-well plate with DMEM (Hyclone) containing 10% FBS (Hyclone). When the cell density reached about 80-85%, the medium remaining in the well was removed and differentiation into myotubes was induced by treating the cells with DMEM (Hyclone) containing 2% HS (Hyclone). The differentiation was performed for a total of 6 days by replacing the medium with a fresh one every two days. After the differentiation, the cells were treated with each of the Cudrania tricuspidata leaf hot water and ethanol extracts prepared in Examples 1-1 to 1-5 at 40 μg/mL, which was dissolved in DMEM (Hyclone) containing 50 ng/mL tumor necrosis factor alpha (TNF-α; PeproTech, Rocky Hills, N.J., USA). 12 hours later, total RNA was isolated using a TRIzol reagent (Takara, Otsu, Japan). The isolated total RNA was quantified using NanoDrop 1000 (Thermo Fisher Scientific Inc., Waltham, Mass., USA). cDNA was synthesized from 16 μL of the quantified RNA using a reverse transcriptase premix (ELPIS-Biotech) and a PCR machine (Gene Amp PCR System 2700; Applied Biosystems, Foster City, Calif., USA) under the condition of 42° C. for 55 minutes, and 70° C. for 15 minutes. PCR was performed by repeating 30 cycles of 95° C. for 30 seconds, 60° C. for 1 minute, and 72° C. for 1 minute using 1 μL of cDNA from 16 μL of the produced cDNA, specific primers described below (Bioneer, Daejeon, Korea) and a PCR premix (ELPIS-Biotech).

MuRF1
(SEQ ID NO 1)
Forward primer: 5′-CCGGACGGAAATGCTATGGA-3′
(SEQ ID NO 2)
Reverse primer: 5′-AGCCTGGAAGATGTCGTTGG-3′
Atrogin-1
(SEQ ID NO 3)
Forward primer: 5′-GTTACTGCAACAAGGAGAATCTGTT-3′
(SEQ ID NO 4)
Reverse primer: 5′-CCGTATGAGTCTTATGTTTTGCTGG-3′
β-Actin:
(SEQ ID NO 5)
Forward primer: 5′-TGACAGGATGCAGAAGGAGATTAC-3′
(SEQ ID NO 6)
Reverse primer: 5′-TAAAACGCAGCTCAGTAACAGTC-3′

The cDNA amplified by the PCR was separated by 1.5% agarose gel electrophoresis, and cDNA bands were identified using the G;BOX EF imaging system (Syngene).

As shown in FIG. 2, the increased mRNA expression of atrogin-1 and MuRF1 by treatment with TNF-α was decreased by the Cudrania tricuspidata leaf hot water and ethanol extracts. This means that the Cudrania tricuspidata leaf hot water and ethanol extracts of the present disclosure have superior ability of inhibiting muscle protein degradation in muscle cells.

### [Example 7] Muscle Protein Degradation-Inhibiting Effect of Cudrania tricuspidata Fruit Hot Water and Ethanol Extracts

Experiment was carried out in the same manner as in Example 6, except that each of the Cudrania tricuspidata fruit hot water and ethanol extracts prepared in Examples 2-1 to 2-5 was treated at 40 μg/mL.

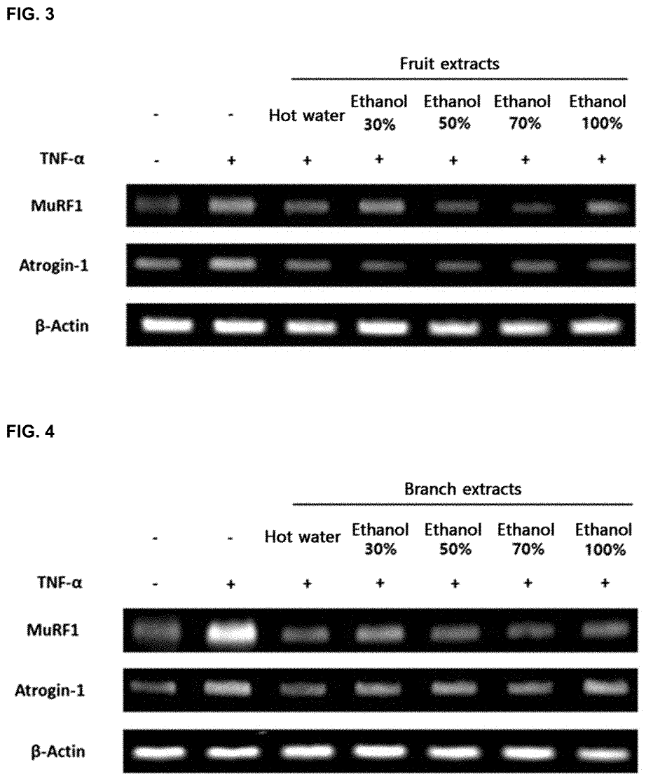

As shown in FIG. 3, whereas the mRNA expression of atrogin-1 and MuRF1 was increased by treatment with TNF-α as compared to the control group, the treatment with the Cudrania tricuspidata fruit hot water and ethanol extracts resulted in the decrease of the mRNA expression as compared to the TNF-α treatment group. This means that the Cudrania tricuspidata fruit hot water and ethanol extracts of the present disclosure have superior ability of inhibiting muscle protein degradation in muscle cells.

### [Example 8] Muscle Protein Degradation-Inhibiting Effect of Cudrania tricuspidata Branch Hot Water and Ethanol Extracts

Experiment was carried out in the same manner as in Example 6, except that each of the Cudrania tricuspidata fruit hot water and ethanol extracts prepared in Examples 3-1 to 3-5 was treated at 40 μg/mL.

As shown in FIG. 4, whereas the mRNA expression of atrogin-1 and MuRF1 was increased by treatment with TNF-α as compared to the control group, the treatment with the Cudrania tricuspidata branch hot water and ethanol extracts resulted in the decrease of the mRNA expression as compared to the TNF-α treatment group. This means that the Cudrania tricuspidata branch hot water and ethanol extracts of the present disclosure have superior ability of inhibiting muscle protein degradation in muscle cells.

### [Example 9] Muscle Protein Degradation-Inhibiting Effect of Cudrania tricuspidata Extracts

Experiment was repeated 3 times in the same manner as in Example 6, except that each of the Cudrania tricuspidata leaf ethyl acetate, hexane, subcritical and supercritical extracts of Examples 1-6 to 1-10, the Cudrania tricuspidata fruit subcritical and supercritical extracts of Examples 2-6 to 2-8 and the Cudrania tricuspidata branch subcritical and supercritical extracts of Examples 3-6 to 3-8 was treated at 40 μg/mL. Then, after measuring the band density of MuRF1 and atrogin-1 mRNAs using the Image J program (National Institute of Health, Bethesda, Md., USA), the measurement result was represented as the mean±standard deviation of percentage (%) with respect to a control group.

As shown in Table 2, whereas the treatment with TNF-α significantly increased the mRNA expression of atrogin-1 and MuRF1 (^{##}p<0.01), the treatment with the Cudrania tricuspidata extracts resulted in significant decrease thereof (**p<0.01). This means that the Cudrania tricuspidata leaf extracts of the present disclosure have superior ability of inhibiting muscle protein degradation in muscle cells.

TABLE 2

| | Relative atrogin-1 | Relative MuRF1 |
|---|---|---|
| Test groups | expression level (%) | expression level (%) |
| TNF-α | 347.6 ± 36.9** | 338.0 ± 26.1** |
| Example 1-6 | 150.7 ± 45.8** | 182.7 ± 31.3** |
| Example 1-7 | 166.7 ± 39.0** | 176.0 ± 36.8** |
| Example 1-8 | 149.3 ± 45.2** | 183.3 ± 29.3** |
| Example 1-9 | 165.8 ± 32.1** | 190.3 ± 23.5** |
| Example 1-10 | 147.1 ± 42.5** | 162.5 ± 34.9** |
| Example 2-6 | 185.0 ± 38.4** | 204.8 ± 20.4* |
| Example 2-7 | 190.6 ± 29.4** | 210.5 ± 30.5** |
| Example 2-8 | 180.7 ± 46.8** | 198.4 ± 28.5** |
| Example 3-6 | 178.9 ± 38.4** | 190.6 ± 40.8** |
| Example 3-7 | 180.4 ± 32,9** | 217.5 ± 36.9** |
| Example 3-8 | 177.2 ± 42.8** | 210.6 ± 38.3** |

### [Example 10] Muscular Function- and Muscle Mass-Improving Effect of Cudrania tricuspidata Leaf 50% Ethanol Extract in Animal Model

<10-1> Animal Breeding and Induction of Muscle Atrophy

7-week-old male mice (C57BL/6J; DBL, Korea) were purchased as experimental animals. The breeding environment was maintained at a temperature of 23±2° C. and a relative humidity of 55±10%. Before the start of the experiment, a total of 20 mice were randomly divided into groups of 5 mice each. After a week of adaptation, anesthesia was induced by intraperitoneal injection of 325 mg/kg tribromoethanol (Sigma-Aldrich). After the anesthesia, the right hind limb gastrocnemius muscle and the right foot pad of the mice in the muscle atrophy group and the sample administration group were stapled using a skin stapler (Unidus, Chungcheongbuk-do, Korea) to damage the muscles and the right hind limp was prevented from moving. This state was maintained for a week. After one week, the staples fixed to the gastrocnemius muscle and the foot pad were removed and the Cudrania tricuspidata leaf 50% ethanol extract of Example 1-3 was orally administered every day for a week at a concentration of 50 mg/kg or 150 mg/kg. For the normal group and the muscle atrophy group, saline was orally administered instead of the sample.

<10-2> Muscle Strength-Improving Effect of Cudrania tricuspidata Leaf 50% Ethanol Extract

After the end of the oral administration period, the muscle strength of the mice was measured using a muscle strength meter (Panlab, Barcelona, Spain). The tail of the mouse was pulled with constant force until the mouse released the bar of the muscle strength meter. A total of 5 consecutive tests were carried out per mouse.

As shown in FIG. 5, whereas the muscle strength was significantly decreased (^{#}p<0.05) in the muscle atrophy group as compared to the normal group, the treatment with the Cudrania tricuspidata leaf 50% ethanol extract at 50 mg/kg or 150 mg/kg resulted in significant increase (*p<0.05, **p<0.01) of muscle strength. This means that the Cudrania tricuspidata leaf 50% ethanol extract of the present disclosure has a superior effect of increasing muscle strength decreased due to muscle atrophy.

<10-3> Muscle Volume-Improving Effect of Cudrania tricuspidata Leaf 50% Ethanol Extract

After anesthetizing the mouse with isoflurane, the volume and density of the right hind limb muscle were measured by positron emission tomography/computed tomography/single photon emission tomography (microPET/CT/SPECT; Siemens Inveon, Knoxville, Tenn., USA).

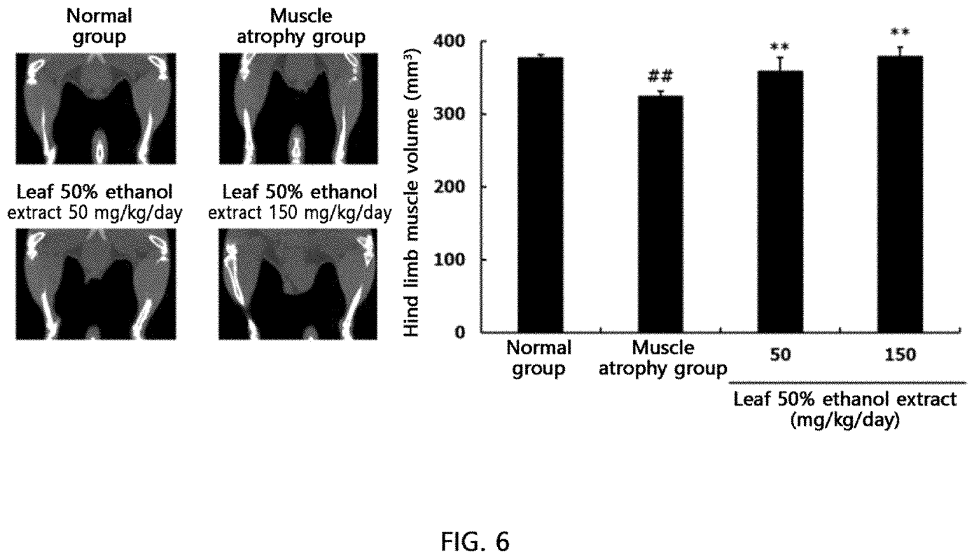

As shown in FIG. 6, whereas the muscle volume was significantly decreased (^{##}p<0.01) in the muscle atrophy group as compared to the normal group, the treatment with the Cudrania tricuspidata leaf 50% ethanol extract at 50 mg/kg or 150 mg/kg resulted in significant increase (^{##}p<0.01) of muscle volume. This means that the Cudrania tricuspidata leaf 50% ethanol extract of the present disclosure has a superior effect of increasing muscle volume decreased due to muscle atrophy.

<10-4> Muscle Weight-Increasing Effect of Cudrania tricuspidata Leaf 50% Ethanol Extract

After the muscle strength measurement was finished, the experimental animal was anesthetized by intraperitoneal injection of 325 mg/kg tribromoethanol (Sigma-Aldrich) and then sacrificed by cardiac puncture. After confirming that heartbeat was stopped, the undamaged tibialis anterior muscle was extracted from the right hind limb and weighed.

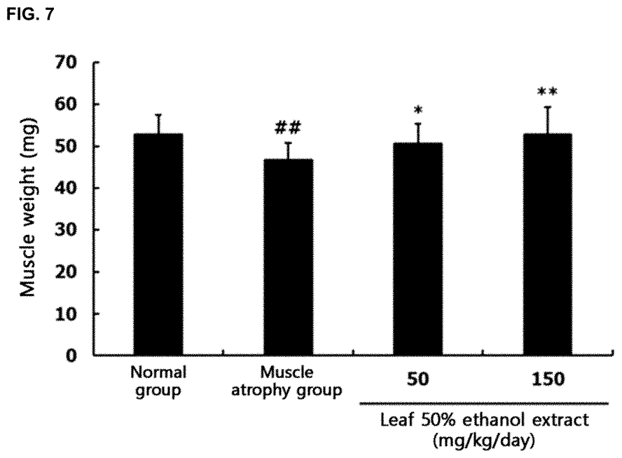

As shown in FIG. 7, whereas the weight of the tibialis anterior muscle was significantly decreased (^{##}p<0.01) in the muscle atrophy group as compared to the normal group, the treatment with the Cudrania tricuspidata leaf 50% ethanol extract at 50 mg/kg or 150 mg/kg resulted in significant increase (^{##}p<0.01; **p<0.01) of muscle weight. This means that the Cudrania tricuspidata leaf 50% ethanol extract of the present disclosure has a superior effect of increasing muscle weight decreased due to muscle atrophy.

### [Example 11] Muscular Function- and Muscle Mass-Increasing Effect of Pulverization Product of Cudrania tricuspidata and Cudrania tricuspidata Hot Water Extract in Animal Model

Muscle strength and muscle weight were measured after administering 500 mg/kg of a Cudrania tricuspidata leaf dried pulverization product and 150 mg/kg of the Cudrania tricuspidata leaf hot water extract of Example 1; 500 mg/kg of a Cudrania tricuspidata fruit dried pulverization product and 150 mg/kg of the Cudrania tricuspidata fruit hot water extract of Example 2; or 500 mg/kg of a Cudrania tricuspidata branch dried pulverization product and 150 mg/kg of the Cudrania tricuspidata branch hot water extract of Example 3 in the same manner as in Example 10.

As shown in Table 2, whereas the muscle strength (^{#}p<0.05) and muscle weight (^{##}p<0.01) were significantly decreased in the muscle atrophy group as compared to the normal group, the muscle strength and muscle weight were significantly increased (*p<0.05, **p<0.01) in all the sample treatment groups as compared to the muscle atrophy group. This means that the pulverization products and hot water extracts of Cudrania tricuspidata leaf, fruit and branch of the present disclosure have a superior effect of increasing muscle strength and muscle weight decreased due to muscle atrophy.

TABLE 3

| Test groups | Muscle strength (g) | Muscle weight (mg) |
|---|---|---|
| Normal group | 196.3 ± 12.9 | 54.7 ± 4.2 |
| Muscle atrophy group | 170.6 ± 10.8^{#} | 43.8 ± 3.3^{##} |
| Cudrania tricuspidata leaf | | |
| pulverization product | 194.9 ± 10.4** | 51.2 ± 4.9** |
| Cudrania tricuspidata leaf | 192.8 ± 8.2** | 53.2 ± 5.3** |
| hot water extract | | |
| Cudrania tricuspidata fruit | 189.5 ± 9.0* | 49.9 ± 4.6* |
| pulverization product | | |
| Cudrania tricuspidata fruit | 190.4 ± 10.5** | 52.8 ± 5.0** |
| hot water extract | | |
| Cudrania tricuspidata branch | 188.6 ± 8.2* | 49.0 ± 3.7* |
| pulverization product | | |
| Cudrania tricuspidata branch | 193.2 ± 10.1** | 53.1 ± 5.1** |
| hot water extract | | |

Hereinafter, preparation examples of pharmaceuticals, foods and cosmetics containing the Cudrania tricuspidata extract according to the present disclosure as an active ingredient are described. However, they are not intended to limit the scope of the present disclosure. The pharmaceutical, food and cosmetic compositions of

Preparation Examples 1-3 were prepared as follows by common methods using the pulverization product of Cudrania tricuspidata or Cudrania tricuspidata extract having superior effect of preventing or treating a muscular disease, or improving muscular function as described above.

### [Preparation Example 1] Pharmaceuticals

<1-1> Powder

After mixing 50 mg of the Cudrania tricuspidata extract of the present disclosure and 2 g of crystalline cellulose, a powder was prepared by filling the mixture in a pouch according to a common method.

<1-2> Tablet After mixing 50 mg of Cudrania tricuspidata, a pulverization product of Cudrania tricuspidata or a Cudrania tricuspidata extract of the present disclosure, 400 mg of crystalline cellulose and 5 mg of magnesium stearate, a tablet was prepared according to a common tableting method.

<1-3> Capsule

After mixing 30 mg of Cudrania tricuspidata, a pulverization product of Cudrania tricuspidata or a Cudrania tricuspidata extract of the present disclosure, 100 mg of whey protein, 400 mg of crystalline cellulose and 6 mg of magnesium stearate, a capsule was prepared by filling the mixture in a gelatin capsule according to a common method.

### [Preparation Example 2] Foods

<2-1> Health Food

1000 mg of the pulverization product of Cudrania tricuspidata or Cudrania tricuspidata extract of the present disclosure, 70 μg of vitamin A acetate, 1.0 mg of vitamin E, 0.13 mg of vitamin B₁, 0.15 mg of vitamin B₂, 0.5 mg of vitamin B₆, 0.2 μg of vitamin B₁₂, 10 mg of vitamin C, 10 μg of biotin, 1.7 mg of nicotinamide, 50 μg of folic acid, 0.5 mg of calcium pantothenate, 1.75 mg of ferrous sulfate, 0.82 mg of zinc oxide, 25.3 mg of magnesium carbonate, 15 mg of monopotassium phosphate, 55 mg of dicalcium phosphate, 90 mg of potassium citrate and 100 mg of calcium carbonate were mixed with 24.8 mg of magnesium chloride. The mixing ratio may be arbitrarily modified. After mixing the above ingredients and preparing a granule therefrom, a health food composition was prepared according to a common method.

<2-2> Health Beverage

After mixing 1000 mg of the Cudrania tricuspidata extract of the present disclosure, 1000 mg of citric acid, 100 g of oligosaccharide, 2 g of plum concentrate and 1 g of taurine and adding purified water to make a total volume of 900 mL, followed by heating for about 1 hour at 85° C. under stirring, the prepared solution was filtered, taken in a sterilized 2-L vessel, sealed, sterilized and then stored in a refrigerator until use for preparation of a health beverage composition.

<2-3> Chewing Gum

A chewing gum was prepared according to a common method by mixing 20 wt % of gum base, 76.9 wt % of sugar, 1 wt % of a flavorant and 2 wt % of water with 0.1 wt % of the Cudrania tricuspidata extract of the present disclosure.

<2-4> Candy

A candy was prepared according to a common method by mixing 60 wt % of sugar, 39.8 wt % of starch syrup and 0.1 wt % of a flavorant with 0.1 wt % of the Cudrania tricuspidata extract of the present disclosure.

<2-5> Biscuit

A biscuit was prepared according to a common method by mixing 25.59 wt % of soft flour, 22.22 wt % of plain flower, 4.80 wt % of refined sugar, 0.73 wt % of table salt, 0.78 wt % of glucose, 11.78 wt % of palm shortening, 1.54 wt % of ammonium, 0.17 wt % of sodium bicarbonate, 0.16 wt % of sodium bisulfite, 1.45 wt % of rice flower, 0.0001 wt % of vitamin B, 0.04 wt % of milk flavor, 20.6998 wt % of water, 1.16 wt % of whole milk powder, 0.29 wt % of substitute milk powder, 0.03 wt % of monocalcium phosphate, 0.29 wt % of spray salt and 7.27 wt % of oil mist with 0.8301 wt % of the pulverization product of Cudrania tricuspidata or Cudrania tricuspidata extract of the present disclosure.

### [Preparation Example 3] Cosmetics

<3-1> Nourishing Lotion (Milk Lotion)

A nourishing lotion was prepared using the Cudrania tricuspidata extract of the present disclosure by a common method according to the composition described in Table 4.

TABLE 4

| Ingredients | Preparation Example 3-1 (wt %) |
|---|---|
| Cudrania tricuspidata extract | 2.0 |
| Squalane | 5.0 |
| Beeswax | 4.0 |
| Polysorbate 60 | 1.5 |
| Sorbitan sesquioleate | 1.5 |
| Liquid paraffin | 0.5 |
| Caprylic/capric triglyceride | 5.0 |
| Glycerin | 3.0 |
| Butylene glycol | 3.0 |
| Propylene glycol | 3.0 |
| Carboxyvinyl polymer | 0.1 |
| Triethanolamine | 0.2 |
| Antiseptic, colorant and flavorant | Adequate |
| Purified water | To 100 |

<3-2> Softening (Skin Lotion)

A softening lotion was prepared using the Cudrania tricuspidata extract of the present disclosure by a common method according to the composition described in Table 5.

TABLE 5

| Ingredients | Preparation Example 3-2 (wt %) |
|---|---|
| Cudrania tricuspidata extract | 2.0 |
| Glycerin | 3.0 |
| Butylene glycol | 2.0 |
| Propylene glycol | 2.0 |
| Carboxyvinyl polymer | 0.1 |
| PEG 12 nonyl phenyl ether | 0.2 |
| Polysorbate 80 | 0.4 |
| Ethanol | 10.0 |
| Triethanolamine | 0.1 |
| Antiseptic, colorant and flavorant | Adequate |
| Purified water | To 100 |

<3-3> Nourishing Cream

A nourishing cream was prepared using the Cudrania tricuspidata extract of the present disclosure by a common method according to the composition described in Table 6.

TABLE 6

| Ingredients | Preparation Example 3-3 (wt %) |
|---|---|
| Cudrania tricuspidata extract | 2.0 |
| Polysorbate 60 | 1.5 |
| Sorbitan sesquioleate | 0.5 |
| PEG 60 hydrogenated castor oil | 2.0 |
| Liquid paraffin | 10 |
| Squalane | 5.0 |
| Caprylic/capric triglyceride | 5.0 |
| Glycerin | 5.0 |
| Butylene glycol | 3.0 |
| Propylene glycol | 3.0 |
| Triethanolamine | 0.2 |
| Antiseptic | Adequate |
| Colorant | Adequate |
| Flavorant | Adequate |
| Purified water | To 100 |

<3-4> Massage Cream

A massage cream was prepared using the Cudrania tricuspidata extract or Cudrania tricuspidata extract of the present disclosure by a common method according to the composition described in Table 7.

TABLE 7

| Ingredients | Preparation Example 3-4 (wt %) |
|---|---|
| Pulverization product | 1.0 |
| of Cudrania tricuspidata | |
| or Cudrania tricuspidata extract | |
| Beeswax | 10.0 |
| Polysorbate 60 | 1.5 |
| PEG 60 hydrogenated castor oil | 2.0 |
| Sorbitan sesquioleate | 0.8 |
| Liquid paraffin | 40.0 |
| Squalane | 5.0 |
| Caprylic/capric triglyceride | 4.0 |
| Glycerin | 5.0 |
| Butylene glycol | 3.0 |
| Propylene glycol | 3.0 |
| Triethanolamine | 0.2 |
| Antiseptic, colorant and flavorant | Adequate |
| Purified water | To 100 |

<3-5> Pack

A pack was prepared using the Cudrania tricuspidata extract or Cudrania tricuspidata extract of the present disclosure by a common method according to the composition described in Table 8.

TABLE 8

| Ingredients | Preparation Example 3-5 (wt %) |
|---|---|
| Pulverization product | 1.0 |
| of Cudrania tricuspidata | |
| or Cudrania tricuspidata extract | |
| Polyvinyl alcohol | 13.0 |
| Sodium carboxymethyl cellulose | 0.2 |
| Glycerin | 5.0 |
| Allantoin | 0.1 |
| Ethanol | 6.0 |
| PEG 12 nonyl phenyl ether | 0.3 |
| Polysorbate 60 | 0.3 |
| Antiseptic, colorant and flavorant | Adequate |
| Purified water | To 100 |

<3-6> Gel

A gel was prepared using the Cudrania tricuspidata extract of the present disclosure by a common method according to the composition described in Table 9.

TABLE 9

| Ingredients | Preparation Example 3-6 (wt %) |
|---|---|
| Cudrania tricuspidata extract | 0.5 |
| Sodium ethylenediaminetetraacetate | 0.05 |
| Glycerin | 5.0 |
| Carboxyvinyl polymer | 0.3 |
| Ethanol | 5.0 |
| PEG 60 hydrogenated castor oil | 0.5 |
| Triethanolamine | 0.3 |
| Antiseptic, colorant and flavorant | Adequate |
| Purified water | To 100 |

We claim:

1-8. (canceled)

9. A method for treating or preventing a muscular disease, or improving muscular function, comprising administering a composition comprising Cudrania tricuspidata as an active ingredient to a subject in need thereof.

10. The method according to claim 9, the Cudrania tricuspidata is a pulverization product of the leaf, fruit or branch of Cudrania tricuspidata, or a Cudrania tricuspidata extract.

11. The method according to claim 10, wherein the Cudrania tricuspidata extract is obtained by extracting the leaf, fruit or branch of Cudrania tricuspidata.

12. The method according to claim 11, wherein the Cudrania tricuspidata extract is obtained by extracting Cudrania tricuspidata with one or more solvent selected from a group consisting of water, a C₁₋₆ organic solvent, a subcritical fluid and a supercritical fluid.

13. The method according to claim 12, wherein the organic solvent is one or more solvent selected from a group consisting of a C₁₋₆ alcohol, acetone, ether, benzene, chloroform, ethyl acetate, methylene chloride, hexane, cyclohexane and petroleum ether.

14. The method according to claim 9, wherein the muscular disease is one or more selected from a group consisting of atony, muscular atrophy, muscular dystrophy, muscle degeneration, muscle weakness, cachexia and sarcopenia.

15. The method according to claim 9, wherein the composition is a food composition.

16. The method according to claim 9, wherein the composition is a pharmaceutical composition.

17. The method according to claim 9, wherein the composition is a cosmetic composition.

18. The method according to claim 9, wherein the composition is a feed additive.
